(19) United States
(12) Patent Application Publication
(10) Pub. No.: US 2011/0076333 A1
(43) Pub. Date: Mar. 31, 2011

(54) METHOD AND COMPOSITIONS FOR SELECTIVELY TREATING SKIN

(76) Inventor: Susan Daly, Basking Ridge, NJ (US)

(21) Appl. No.: 12/567,308

(22) Filed: Sep. 25, 2009

(51) Int. Cl. A61K 8/64; A61Q 1/02; A61K 8/02; A61K 8/72

(52) U.S. Cl. 424/489; 424/63; 424/78.03; 424/70.14; 514/20.7

(57) ABSTRACT

This invention relates to compositions, methods and kits for selectively depositing a benefit agent on skin without depositing the benefit agent on the hair. The method relates to exposing the skin to a composition containing anionic proteins, anionic polymers, anionic dyes, anionic pigments, or mixtures thereof which have an isoelectric point of less than about 4.5, the compositions having a pH of about 3.5 to about 5.5. In accordance with the methods of this invention, the benefit agent becomes deposited on the skin; however, any hair that is also contacted by the benefit compositions remains substantially untreated.

### FIELD OF THE INVENTION

This invention relates to a one-step method for selectively depositing a treatment agent such as a coloring agent or another active or benefit agent on mammalian skin without simultaneously depositing said agent on hair. The method of this invention relates to treating the skin with a composition containing a benefit agent, said benefit agent having an isoelectric point of less than about 4.5 and, more preferably, less than about 3.5. The benefit agent is preferably dispersed in a buffering system having a pH of about 3.5 to about 5.5 with low salt concentrations. In accordance with the methods of this invention, the benefit agent is deposited on skin without being deposited on hair that comes into contact with the benefit agent.

### BACKGROUND OF THE INVENTION

For many years, individuals have used cosmetic care and treatment compositions on their skin in an effort to protect, enhance and/or change certain characteristics of their skin, such as make-up applications, moisturizers and tanning and sunscreens for protection from environmental effects. Most of these care and treatment compositions function either by coating the skin with a treatment composition, as with conditioning agents, or by a pigment particle product applied to the surface of the skin, as in cosmetic or make-up applications. However, most skin care products and treatment compositions are not selective to skin; that is, they also have some effect on other surfaces, such as hair, with which they come into contact when they are being applied to the skin. In some cases, this effect on hair is undesirable and care must be taken to limit contact of the treatment composition to the skin only and to avoid contact with the hair.

Existing skin make-up applications generally rely on the use of pigment particles to apply a color to the surface of the skin. Such pigments applied to hair would not be aesthetically pleasing. If this occurs, users must try to wash the color off the hair while maintaining the color on the skin.

Thus, heretofore, there has not been means for selectively applying benefit agents to skin by sustainably attaching treatment agents to the skin while simultaneously substantially preventing attachment of the treatment agents to the hair.

### SUMMARY OF THE INVENTION

Surprisingly, it has been found that exposure of the skin and hair to a benefit agent dispersed within a buffering system having a pH of about 3.5 to about 5.5, said benefit agent having an isoelectric point of less than about 4.5, said composition preferably having a low ionic strength, results in selective deposition of the benefit agent onto skin without simultaneously depositing the benefit agent onto hair. Buffer salts, other inorganic salts added to the composition (such as NaCl or KCl), and other charged components of the composition contribute to the total ionic strength of the solution.

The method of this invention relates to a means and kit for selectively depositing treatment agents on skin. The methods of this invention preferably include the following steps:

a) providing a composition containing a benefit agent selected from the group consisting of an anionic peptide, an anionic polymer, an anionic protein, an anionic dye, an anionic pigment and a combination thereof, wherein the benefit agent has an isoelectric point of less than about 4.5 and said composition has a pH of about 3.5 to about 5.5; and

b) applying the composition to mammalian skin for a time period sufficient for at least one of said benefit agents to be deposited on the skin and form a layer.

The application of the compositions of this invention may be repeated one or more times to deposit selectively additional layers of benefit agents onto the skin. Such additional layers may provide enhanced treatment and improved resistance to removal by surfactant treatment or cleansing.

Other features and advantages of this invention will be apparent from the detailed description of the invention and from the claims.

### DETAILED DESCRIPTION OF THE INVENTION

The method and kits of this invention provide selective deposition of isoelectric benefit agents to skin, without deposition of said benefit agents onto exposed hair, by deposition of an isoelectrically charged benefit agent within a composition having a preferable pH range. Unexpectedly, we found that controlling the isoelectric point and the pH of the benefit agent from which the isoelectrically charged agents were deposited resulted in deposition of benefit agent only onto the skin, leaving the hair free of deposition. Thus, the methods of this invention may be used for selectively depositing benefit agents, (including, but not limited to peptides, polymers, proteins, hair dyes, pigments and the like), onto skin and not hair.

A one-step method of this invention involves providing a composition containing a benefit agent having a maximum isoelectric point, which may be an anionic particle or other benefit agent, and applying the composition to skin to form a first layer on the skin. The composition preferably has a pH of between about 3.5 and about 5.5, more preferably between about 4.0 and about 5.0 and most preferably between about 4.25 and about 4.75. Due to its isoelectric characteristics, the said first layer will not substantially form on any hair that is exposed to the composition simultaneously with exposure of the composition to the skin. Without wishing to be bound by theory, it is believed that the reason for the selective deposition of the composition lies in the differences in the surface properties, including surface charge, of the skin and the hair.

We believe that one skilled in the art can, based upon the description herein, utilize the compositions and methods of this invention to their fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

### DEFINITIONS

“Skin”, as used herein, relates to any of the outer, epidermal covering of the body, including, without limitation, the scalp, the lips, and the mucous membranes.

“Hair”, as used herein, relates to any keratin-containing fiber or fibrous material, including any human scalp or body hair, animal hair, wool, and fur.

“Isoelectric point”, relates to the pH of a solution in which a charged molecule does not migrate in an electric field.

“Anionic”, as used herein, is used to describe a compound or material with a negative charge. Such compounds generally move toward the positive electrode in electrolysis.

“Ionic strength” as used herein, relates to the concentration of ions dissolved in the solution.

“Peptide”, as used herein, is a molecule containing two or more amino acids joined by a peptide bond or modified peptide bonds.

The term “amino acid” refers to the basic chemical structural unit of a protein or polypeptide. The following abbreviations are used herein to identify specific amino acids:

TABLE 1

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Protein”, as used herein, relates to a long chain of amino acids joined together by peptide bonds and having a molecular weight of greater than 10,000 Da.

“Polymer”, as used herein, relates to a large organic molecule formed by combining many smaller molecules (monomers) in a regular pattern.

“Benefit agent”, as used herein, relates to any compound that may be used to produce a desirable cosmetic effect on skin.

“Particle”, as used herein, refers to a small, discrete portion of material that has mass and dimension.

“Microparticle”, as used herein, refers to a particle having a diameter ranging from about 1 to about 1000 micrometers.

“Nanoparticle”, as used herein, refers to a particle having a diameter ranging from about 1 to about 1000 nanometers.

“Pigment”, as used herein, refers to a fine, insoluble white, black or colored material. For the purposes of this invention, pigments also include pigment particles, pigment microparticles, and pigment nanoparticles.

“Molar ratio”, as used herein, relates to the ratio of the molecular weight of a specific portion of a molecule to the molecular weight of the whole molecule.

“Diameter”, as used herein, refers to the largest side-to-side linear dimension of a particle, microparticle, or nanoparticle.

“Zeta potential”, as used herein, relates to an electrokinetic measurement in a colloidal system. Zeta potential is the difference in potential between the dispersion medium and the immovable layer attached to the surface of the dispersed phase. Zeta potential may be measured using a Malvern zetasizer.

### Anionic Benefit Agents

The anionic benefit agents contained in the composition of this invention can be any anionically charged compounds useful for imparting a cosmetically desirable effect on skin. Nonlimiting examples include anionic proteins, anionic peptides, anionic polymers, anionic particles, anionic dyes and mixtures of these.

Anionic proteins include naturally-occurring anionic proteins and synthetic anionic proteins. Examples of naturally-occurring anionic proteins include, without limitation, wheat acidic esterase; alkaline phosphatase; beta-galactosidase; lactase; lipase; amylases; Epidermal Growth Factor; glycosidases; glucose oxidase; nitrate reductase; catalase; lactoglobulin; carboanhydrase; casein proteins from milk; trypsin inhibitor; albumin; anionic proteases, such as cathepsin; proteins from egg white, including ovalbumin, gamma-globulin, and ovomucin.

Synthetic anionic proteins include, for example, polyglutamic acid, polyaspartic acid, copolymers of these materials, and proteins containing a molar ratio of 50% or more of glutamic acid or aspartic acid amino acids.

Examples of anionic peptides include, without limitation, polyglutamic acid, polyaspartic acid, copolymers of these materials, and peptides containing a molar ratio of 50% or more of glutamic acid or aspartic acid amino acids.

Anionic polymers include naturally-occurring anionic polymers and synthetic anionic polymers. Examples of naturally-occurring anionic polymers include, without limitation, alginic acid, propylene glycol alginate, carrageenan gum, cellulose gum, gum acacia, karaya gum, xanthan gum, tragacanth gum, hyaluronic acid, shellac, and mixtures of these.

Nonlimiting examples of synthetic anionic polymers include sodium laureth sulfate (SLES), sodium polystyrene sulfonate, sodium polymethacrylate, sodium polynapthalenesulphonate, acrylates/C10-30 alkyl acrylate crosspolymer, acrylates/beheneth-25 methacrylate copolymer, acrylates/steareth-20 methacrylate copolymer, acrylates/VA crosspolymer, acrylic acid/acrylonitrogens copolymer, carbomerPVM/MA decadiene crosspolymer, acrylates copolymer, octylacrylamide/acrylates/butylaminoethylmethacrylate copolymer, PVM/MA copolymer, VA/crotonates/vinyl neodecanoate copolymer, glyceryl polymethacrylate, and mixtures of these.

Non-limiting examples of anionic dyes contained in the composition of this invention may include FD&C Red 40, FD&C Yellow 6, FD&C Red #4, Tartrazine, Orange B and mixtures thereof.

The anionic particles contained in the composition of this invention can be anionic pigments, other anionic particles, such as microparticles or nanoparticles, or combinations of these.

The anionic pigments contained in the composition of this invention can be anionically charged colored pigment particles, microparticles or nanoparticles, or combinations of these. Preferred for use in this invention are anionic colored pigments.

Pigments, particularly metal compounds or semimetallic compounds, may be used in the compositions and methods of this invention in ionic, nonionic or oxidized form. The pigments can be in this form either individually or in admixture or as individual mixed oxides or mixtures thereof, including mixtures of mixed oxides and pure oxides. Examples are the titanium oxides (for example TiO₂), zinc oxides (for example ZnO), aluminum oxides (for example Al₂O₃), iron oxides (for example Fe₂O₃), manganese oxides (for example MnO), silicon oxides (for example SiO₂), silicates, cerium oxide, zirconium oxides (for example ZrO₂), barium sulfate (BaSO₄) or mixtures thereof. Suitable pigments are commercially available. An example is Hombitec® L5 (INCI name: titanium dioxides) supplied by Merck.

Other examples of pigments include the following: D&C Red No. 36, D&C Red No. 30, D&C Orange No. 17, Green 3 Lake, Ext. Yellow 7 Lake, Orange 4 Lake, Red 28 Lake, the calcium lakes of D&C Red Nos. 7, 11, 31 and 34, the barium lake of D&C Red No. 12, the strontium lake D&C Red No. 13, the aluminum lakes of FD&C Yellow No. 5 and No. 6, the aluminum lakes of FD&C No. 40, the aluminum lakes of D&C Red Nos. 21, 22, 27, and 28, the aluminum lakes of FD&C Blue No. 1, the aluminum lakes of D&C Orange No. 5, the aluminum lakes of D&C Yellow No. 10; the zirconium lake of D&C Red No. 33, Cromophthal® Yellow, Sunfast® Magenta, Sunfast® Blue, iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, titanium dioxide nanoparticles, zinc oxide, barium oxide, ultramarine blue, bismuth citrate, hydroxyapatite, zirconium silicate, carbon black particles and the like.

The pigments or particles of this invention are anionically charged, either inherently or by virtue of an anionic coating. Suitable anionic coatings include, for example, silica, aluminosilicate, sodium C14-16 olefin sulfonate, disodium stearoyl glutamate, sodium stearoyl glutamate/sodium trideceth-6 carboxylate, and sodium polyacrylates/hydrogenated lecithin/aluminum hydroxide. Examples of anionically coated pigments suitable for use in the present invention are given in Table 2.

TABLE 2

| | Chemical | Surface | |
|---|---|---|---|
| Pigment Name | Name | Coating | Source |
| Sympholight RW | Iron Oxide | silica | Presperse, Inc. |
| | | treated | Somerset, NJ |
| Sympholight BW | Iron Oxide | silica | Presperse, Inc. |
| | | treated | Somerset, NJ |
| SP-4405 Surface | Iron Oxide | alumino- | Color |
| Passivated Black | | silicate | Techniques, |
| Oxide | | | Inc., South |
| | | | Plainfield NJ |
| SP—Surface | Titanium | alumino- | Color |
| Passivated | Dioxide | silicate | Techniques, |
| titanium dioxide | | | Inc., South |
| | | | Plainfield NJ |
| Sinert BP-10 | Iron Oxide | silica | Kobo Products, |
| | | | Inc. South |
| | | | Plainfield NJ |
| Sinert RP5-10 | Iron Oxide | silica | Kobo Products, |
| | | | Inc. South |
| | | | Plainfield NJ |
| BRO-C5 | Iron Oxide | silica | Kobo Products, |
| | | | Inc. South |
| | | | Plainfield NJ |
| Aquaspersabil RIO | Iron Oxide | sodium C₁₄₋₁₆ | Presperse, Inc. |
| | | Olefin | Somerset, NJ |
| | | Sulfonate | |
| Amino Acid (NAID) | Iron Oxide | Disodium | U.S. Cosmetics |
| Treated Iron Oxide | | Stearoyl | Corporation, |
| | | Glutamate | Dayville CT |
| BLACK BL-100 SPA | Iron Oxide | Sodium | Kobo Products, |
| | | Stearoyl | Inc. South |
| | | Glutamate | Plainfield NJ |
| | | (And) Sodium | |
| | | Trideceth-6 | |
| | | Carboxylate | |
| PALI Treated iron | Iron Oxide | Sodium | U.S. Cosmetics |
| Oxide | | Polyacrylates/ | Corporation, |
| | | Hydrogenated | Dayville CT |
| | | Lecithin/ | |
| | | Aluminum | |
| | | Hydroxide | |

The anionic pigments and particles preferred for use in this invention preferably have an Isoelectric Point of less than about 4.5, and preferably less than about 3.5.

The anionic pigments and particles useful in the compositions and methods of this invention preferably have a concentration range of about 0.0005% to about 10% by weight and most preferably from about 0.025% to about 0.5% by weight.

### Buffers

The pH of the compositions of this invention is preferably about 3.5 to about 5.5. Any cosmetically acceptable buffers may be used to adjust the pH of the composition to this desired range. Examples of such buffers include, without limitation, sodium citrate, 2-(N-morpholino) ethanesulfonic acid. Acetate, phosphate, lactate and other buffers known to a person skilled in the art having a buffer capacity between a pH of 3 and 6 are also useful in the compositions of this invention. Other salts, such as sodium chloride or potassium chloride may be added to the buffer. Preferably, the total ionic strength of the composition should be less than about 150 mM and preferably less than about 25 mM.

### Compositions

The compositions of this invention may be prepared in the form of formulations known to be useful for cosmetic skin products. For example, they can be in the form of moisturizers, cleansing agents, tanning products, sunscreens, lotions, dispersions, emulsions, gels, cream gels, creams, pastes, sticks, suspensions, sprays, aerosols, foams, or the like. To the compositions of the invention may be added other substances, auxiliary agents, for example those commonly used for cosmetic products in general. Such materials include, for example, thickeners (for example clays, starches, polyacrylic acid and the derivatives thereof), cellulose derivatives, lanolin derivatives, vitamins or provitamins, (for example biotin, vitamin C, tocopherols or D-panthenol), antigrease agents, inorganic or organic acids (for example lactic acid, citric acid, glycolic acid or phosphoric acid), preservatives (for example para-hydroxybenzoate esters), nonaqueous solvents, antioxidants (for example tocopherols or the esters thereof), dyes and fragrances or perfumes, UV light-absorbing inorganic particles and others known to those of ordinary skill in the art.

### Other Cosmetic Components and Additives

In addition to the above-described ingredients, other common cosmetic components and additives may be incorporated in the compositions of this invention, as long as the basic properties of the compositions, including pH and salt concentration, are preserved. Such ingredients include, but are not limited to, humectants, emollients, moisturizers, inorganic salts, fragrances, hydrotropes, foam stabilizers, preservatives, water softening agents, and the like. Optional components may be present in weight percentages of less than about 2% each, and from about 5% to about 10% by weight of the composition in total.

### Cosmetically Acceptable Carriers:

The compositions of this invention preferably contain one or more cosmetically-acceptable carriers. Preferably, such carriers include water. Organic solvents may also be included in order to facilitate manufacturing of the composition or to provide esthetic properties, such as viscosity control. Suitable solvents include the lower alcohols like ethyl alcohol and isopropyl alcohol; glycol ethers, like 2-butoxyethanol, ethylene glycol monoethyl ether, propylene glycol and diethylene glycol monoethyl ether or monomethyl ether; and mixtures thereof. Non-aqueous solvents may be present in the compositions of the present invention in an amount of about 1% to about 50%, and in particular about 5% to about 25%, by weight of the total weight of the carrier in the composition.

The compositions of this invention should be stable to phase or ingredient separation at a temperature of about 25° C. for an indefinite period of time, or at least for 5 weeks at a temperature of 45° C. Thus, the compositions of this invention have demonstrated sufficient stability to phase and ingredient separation at temperatures normally found in commercial product storage and shipping to remain unaffected for periods of at least one year.

The compositions of this invention may be utilized in any types of products that impart benefits to skin, including, but not limited to the following: skin conditioners, skin moisturizers, tanning products, sunscreens, skin treatment compositions, foundations, make-up compositions and the like.

Treating the skin with the compositions of this invention is generally carried out in accordance with the following steps: applying to dry or wet skin an effective amount of the composition of the invention; (2) distributing the composition of this invention more or less evenly on the skin such that it contacts all the skin or other substrate which is intended to be treated. This permits the anionic compound of the compositions of this invention to be applied thoroughly and evenly on the skin or other substrate to form a layer on the skin. This step may be accomplished by rubbing the composition on the skin manually or using a skin care appliance such as a cloth for up to about 20 minutes; and (3) rinsing said skin or other substrate with water so as to remove excess material that has not deposited onto the skin;

The compositions and methods of this invention are further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

Skin samples used for testing in these examples consisted of tape samples each having a layer of human epidermal cells adhered thereto. The tape samples were prepared by adhering a clean sample of tape to the skin on a human forearm then removing and performing this step twice in order to obtain a second layer of skin. Upon removal, the outer layer of epidermal cells on the skin remained adhered to the tape adhesive. All testing solutions were applied to the tape samples to measure their effect on the adhered skin cells.

All human hair used in the examples of this invention was natural white hair in 250 mg tress samples. Such hair is available commercially, for example from International Hair Importers and Products (Bellerose, N.Y.), and is also available in different colors, such as brown, black, red, and blonde, and in various types, such as African-American, Caucasian, and Asian.

### Example 1

A red iron oxide pigment with a silica coating having an isoelectric point of approximately 3.5 was incubated with either 100% gray hair or tape-stripped stratum corneum for 30 minutes. 250 mg hair tresses were incubated with 10 mL of pigment dispersion. The tape-stripped SC was assembled into a custom build Teflon holder and incubated with 2 mL of pigment dispersion. The pigment dispersions were created at 0.05 wt % in the various buffers specified in Table 3 below. The data set forth in Table 3 demonstrates that the deposition (on a scale from 0 to 5 where 0 is no color deposition and 5 is intense color deposition) of pigment onto hair is undetectable in all cases, while there is a significant amount of deposition onto the TSSC for low ionic strength buffers ranging in pH from 4-5. In a high salt pH 5 buffer, the deposition onto skin is reduced, but we still preserve some selectivity for skin not hair.

TABLE 3

| Buffer | Deposition on Hair | Deposition on Skin |
|---|---|---|
| 10 mM MES, pH 5 | 0 | 2 |
| 5 mM sodium citrate, pH 4.5 | 0 | 2 |
| 5 mM Tris-HCl, pH 7.5 | 0 | 0 |
| 25 mM Tris + 150 mM | 0 | 0 |
| NaCl, pH 7.5 | | |
| 10 mM sodium citrate + | 0 | 1 |
| 150 mM NaCl, pH 5 | | |

### Example 2

A red iron oxide pigment with a silica coating (having an isoelectric point of approximately 3.5 was incubated with either 100% gray hair or tape-stripped stratum corneum for 30 minutes. 250 mg hair tresses were incubated with 10 mL of pigment dispersion. The tape-stripped SC was assembled into a custom build Teflon holder and incubated with 2 mL of pigment dispersion. The pigment dispersions were created at 0.25 wt % in the various buffers specified in Table 4 below. The data set forth in Table 4 demonstrates that deposition (on a scale from 0 to 5 where 0 is no color deposition and 5 is intense color deposition) of pigment onto hair is undetectable in all cases, while there is a significant amount of deposition onto the TSSC for low ionic strength buffers ranging in pH from 4-5. In high salt pH 5 buffer, some of the selectivity is lost, with little pigment deposition onto skin and a small amount of pigment deposition onto hair.

TABLE 4

| Buffer | Deposition on Hair | Deposition on Skin |
|---|---|---|
| 10 mM sodium citrate, pH 5 | 0 | 3 |
| 5 mM sodium citrate, pH 4 | 0 | 4 |
| 5 mM sodium citrate, pH 4.5 | 0 | 4 |
| 5 mM sodium citrate, pH 5 | 0 | 3 |
| 5 mM tris-HCl, pH 7.5 | 0 | 0 |
| 25 mM Tris + 150 mM | 0 | 0 |
| NaCl, pH 7.5 | | |
| 10 mM sodium citrate + | 1 | 1 |
| 150 mM NaCl, pH 5 | | |

The specification and embodiments above are presented to aid in the complete and non-limiting understanding of the invention disclosed herein. Since many variations and embodiments of the invention can be made without departing from its spirit and scope, the invention resides in the claims hereinafter appended.

What is claimed is:

1. A method for selectively depositing materials onto skin of a mammal and not onto hair of a mammal comprising:
a) providing a cosmetic composition comprising at least one anionic material having an isoelectric point of less than about 4.5, wherein said cosmetic composition has a pH of about 3.5 to about 5.5;
b) applying said cosmetic composition to skin for a time period sufficient for at least one said anionic material to be deposited on the skin and form a layer, whereby said anionic material is deposited on the skin of a mammal and not on the hair of a mammal.

2. A method according to claim 1 wherein said anionic material is selected from the group consisting of an anionic peptide, an anionic polymer, an anionic protein, an anionic dye, an anionic pigment and a combination thereof.

3. A method according to claim 1, wherein said anionic material has an Isoelectric Point of less than about 4.5.

4. A method according to claim 3, wherein said anionic material has an Isoelectric Point of from less than about 3.5.

5. A method according to claim 2, wherein said anionic peptide comprising polyglutamic acid, polyaspartic acid, copolymers of these materials, and peptides containing a molar ratio of 50% or more of glutamic acid or aspartic acid amino acids.

6. A method according to claim 2, wherein said anionic material is a peptide selected from the group consisting of polylysine, polyarginine and the mixtures thereof.

7. A method according to claim 1, wherein said anionic material is a pigment selected from the group consisting of D&C Red No. 36, D&C Red No. 30, D&C Orange No. 17, Green 3 Lake, Ext. Yellow 7 Lake, Orange 4 Lake, Red 28 Lake, the calcium lakes of D&C Red Nos. 7, 11, 31 and 34, the barium lake of D&C Red No. 12, the strontium lake D&C Red No. 13, the aluminum lakes of FD&C Yellow No. 5 and No. 6, the aluminum lakes of FD&C No. 40, the aluminum lakes of D&C Red Nos. 21, 22, 27, and 28, the aluminum lakes of FD&C Blue No. 1, the aluminum lakes of D&C Orange No. 5, the aluminum lakes of D&C Yellow No. 10; the zirconium lake of D&C Red No. 33, Cromophthal® Yellow, Sunfast® Magenta, Sunfast® Blue, iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, titanium dioxide nanoparticles, zinc oxide, barium oxide, iron oxide coated with inorganic polymer, ultramarine blue, bismuth citrate, hydroxyapatite, zirconium silicate, and carbon black particles.

8. A method according to claim 2, wherein said anionic material is an anionic nanoparticle.

9. A method according to claim 8, wherein said anionic nanoparticle has a diameter of from about 1 to about 1000 nanometers.

10. A method according to claim 1, wherein said cosmetic composition has a pH of about 4.5.

11. A method according to claim 1, wherein said hair is selected from the group consisting of hair, wool and fur.

12. A method according to claim 1, wherein said anionic material is a peptide having a concentration of about 0.000001% to about 10% by weight.

13. A method according to claim 12, wherein said peptide has a concentration of from about 0.001% to about 5% by weight.

14. A method of claim according to claim 13, wherein said peptide has a concentration of about 0.01% to about 2% by weight.

15. A method according to claim 2, wherein said anionic pigment has a concentration of about 0.0005% to about 10% by weight.

16. A method according to claim 15, wherein said anionic pigment has a concentration of about 0.025% to about 0.5% by weight.

17. A method according to claim 1, wherein the ionic strength of the cosmetic composition is less than about 150 mM.

18. A method according to claim 17, wherein the ionic strength of the first cosmetic composition is less than about 25 mM.

19. A cosmetic kit for selectively coloring skin comprising:
a) a container containing a cosmetic composition having a pH of from about 3.5 to about 5.5, comprising at least one anionic material having an isoelectric point of less than about 4.5;
wherein said composition is applied to the skin for a time period sufficient for at least one said anionic material to be deposited on the skin and form a layer, and rinsed off with water.
